(19) United States
(12) Patent Application Publication
(10) Pub. No.: US 2022/0409861 A1
(43) Pub. Date: Dec. 29, 2022

(54) SYSTEM AND METHOD FOR A CATHETER/PROBE POSITIONING AND STABILIZATION CLAMP

(71) Applicant: Chance Witt, Mobile, AL (US)

(72) Inventor: Chance Witt, Mobile, AL (US)

(21) Appl. No.: 17/357,253

(22) Filed: Jun. 24, 2021

(51) Int. Cl. A61M 25/02

(52) U.S. Cl. CPC A61M 25/02; A61B 5/6852

(57) ABSTRACT

A system and method for assisting medical professionals while controlling catheters/probes is provided. The device generally comprises an attachment device, top piece, bottom piece, hinge, locking element, and friction producing pads. A medical professional may manipulate the device in a way that allows for better control of a catheter/probe in at least one direction. The device is configured to be contained within a sterile packaging until ready for use. In some embodiments, the device may be attached to a patient's drape during a procedure so that the catheter/probe may be properly aligned with a sheath used during a procedure.

### FIELD OF THE DISCLOSURE

The subject matter of the present disclosure refers generally to a clamp that can be used to help control movement of cardiac catheters/probes.

### BACKGROUND

Cardiac catheters are used during cardiac procedures for diagnosis and treatment of various medical conditions related to heart disease. These catheters/probes are generally long narrow cylindrical devices—including, but not limited to, electrical wires covered with insulation, intravascular imaging probes, and plastic tubes—and are typically inserted at a point in the groin, such as the femoral artery or vein, and pushed up to the heart. Medical professionals often manually move these devices to their desired location in a patient's body since this provides tactile feedback that the medical professional may use to ensure they are not pushing against any critical structures. Therefore, free movement of devices while moving through a patient is desirable since medical professionals can quickly adjust said devices if the situation requires. However, this free movement of the catheter is in fact not always a desirable trait. Many medical procedures are performed which require catheters/probes to be placed in specific locations within the body for a specified period of time or until the procedure is completed.

Unfortunately, unless a catheter/probe is being held at all times, it can move quite easily. For instance, events ranging from a slight torque on the catheter, heart movement, breathing, and patient movement, can all cause displacement of the catheter/probe and require it to be moved back into position before a medical procedure can be completed, thus slowing the procedure and/or putting a patient at greater risk. Furthermore, some catheters/probes are simply prone to rotating along the long axis, wherein said movement seemingly causes devices such as the intracardiac echocardiography catheter to lose a desired image no matter how slight. No good solution currently exists for this problem besides a medical professional holding the catheter/probe in place manually, which makes it more difficult or even impossible for them to perform other tasks they need to accomplish. And in certain scenarios, medical professionals must create a makeshift torquing device using objects like a hemostat clamp and piece of gauze or a wet towel to help maintain position.

Accordingly, there is a need in the art for a disposable clamp that can be used by a medical professional performing a medical procedure using catheters/probes, wherein said disposable clamp increases a medical professional's control of said catheter/probe while liberating said medical professional from the burden of constantly holding said catheter/probe, allowing said medical professional to accomplish other tasks they may otherwise have been unable to perform.

### SUMMARY

A system and method for assisting medical professionals while controlling catheters/probes is provided. In one aspect, the system is a sterilized clamp configured to be secured to a stationary object and hold a catheter/probe in a desired position. In another aspect, the system is a sterilized clamp that can be used to limit movement of a catheter/probe in a desired direction. The system generally comprises an attachment device, top piece, bottom piece, hinge, locking element, and friction producing pads. In some embodiments of the system, the top piece and locking element may be constructed with multiple latching points used to create a sterile clamping device with incremental levels of compression to provide greater force if necessary and to allow for easy accommodation of various sizes of catheters/probes with the same device. In another embodiment, the material of the pads may have a grain, or small ridges, that provide friction in substantially one direction, but not another, to make control of catheters/probes easier for medical professionals when using the clamp described herein.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTION OF THE DRAWINGS

These and other features, aspects, and advantages of the present disclosure will become better understood with regard to the following description, appended claims, and accompanying drawings where:

FIG. 1 illustrates a positioning and stabilization (PAS) clamp embodying features consistent with the principles of the present disclosure.

FIG. 2 illustrates a PAS clamp embodying features consistent with the principles of the present disclosure.

FIG. 3 illustrates a PAS clamp embodying features consistent with the principles of the present disclosure.

FIG. 4 illustrates a PAS clamp embodying features consistent with the principles of the present disclosure.

FIG. 5 illustrates a PAS clamp embodying features consistent with the principles of the present disclosure.

FIG. 6 illustrates a PAS clamp embodying features consistent with the principles of the present disclosure.

FIG. 7 illustrates a PAS clamp being used within an environment by a medical professional.

FIG. 8 illustrates a PAS clamp being used within an environment by a medical professional.

FIG. 9 illustrates how a PAS clamp, catheter/probe, and sheath may be used together.

FIG. 10 is a flow chart illustrating certain method steps of a method embodying features consistent with the principles of the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally. Although the description generally refers to a clamp used to assist a medical professional control a catheter/probe, one with skill in the art will understand that the system may be used to control other tubular devices without departing from the inventive subject matter described herein.

The term “comprises” and grammatical equivalents thereof are used herein to mean that other components, steps, etc. are optionally present. For example, a system “comprising” components A, B, and C can contain only components A, B, and C, or can contain not only components A, B, and C, but also one or more other components. The term “medical professional” and grammatical equivalents thereof are used herein to mean people working in the healthcare industry. For instance, a medical professional may include a doctor, nurse, nurse practitioner, physician's assistant, radiation technologist, etc. The term “sterile packaging” and grammatical equivalents thereof are used herein to mean the minimum packaging configuration that provides a microbial barrier and allows aseptic presentation of the product unit at the point of use. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

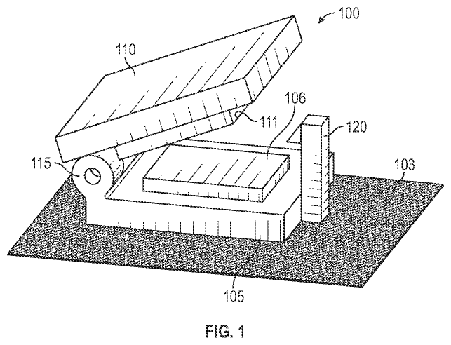

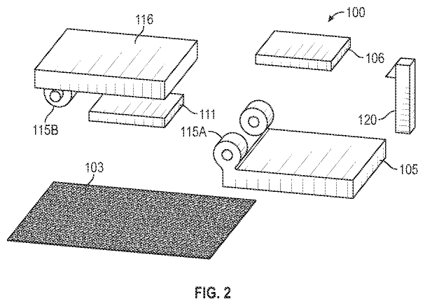

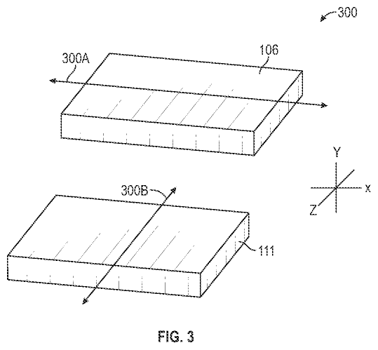

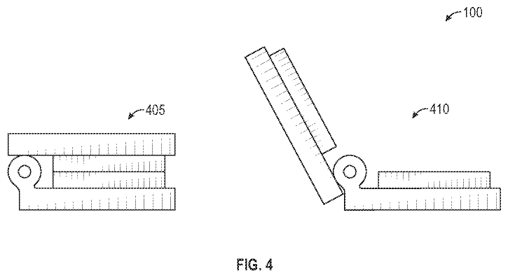

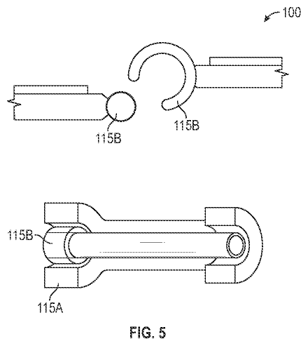

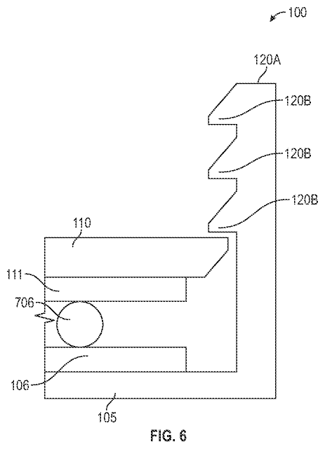

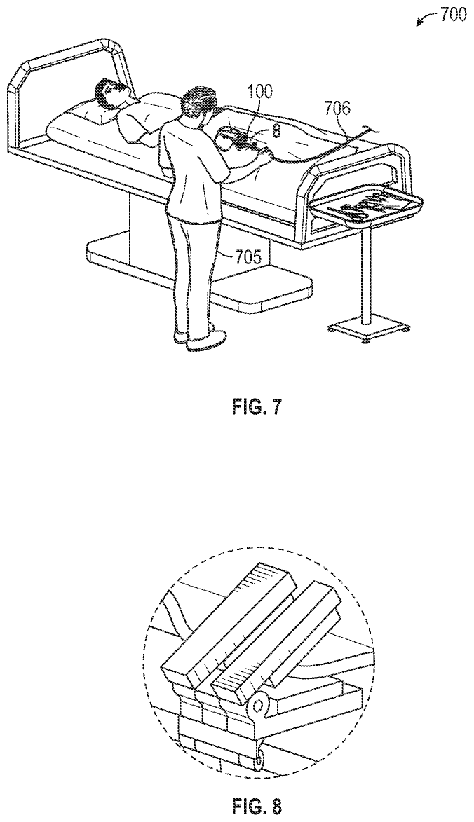

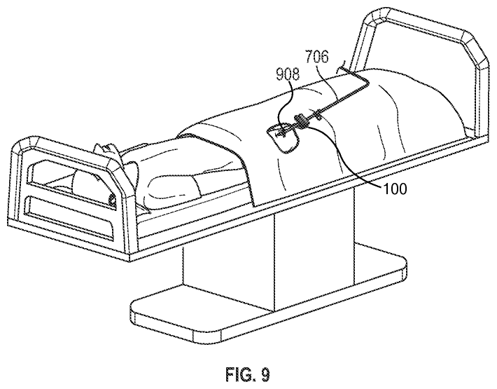

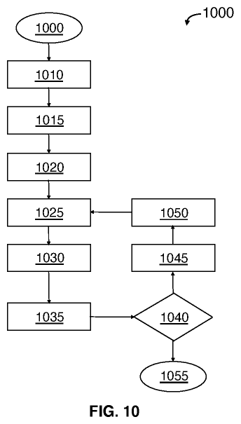

FIGS. 1-10 illustrate embodiments of a positioning and stabilization (PAS) clamp and a method of use. FIG. 1 depicts a perspective view of a preferred embodiment of a device configured to assist medical professionals control movement of a catheter/probe 706. FIG. 2 depicts an exploded view of an embodiment of a device configured to assist medical professionals control movement of a catheter/probe 706. FIG. 3 depicts how the top pad 111 and bottom pad 106 may be configured to have textures that provide a friction force 300 substantially in a single direction, which may give medical professionals more control over movement of a catheter/probe 706. FIG. 4 depicts an embodiment of the PAS clamp 100 in a closed position 405 and an open position 410. FIG. 5 depicts a preferred embodiment of a hinge having a female half having female members and a male half having male members, wherein said male members are configured to fit into said female members via an opening on a lateral side of said female members. FIG. 6 depicts a preferred embodiment of a locking element 120 having a plurality of locking steps 120B designed to engage a top surface of the top piece 110 in order to hold the top piece 110 and bottom piece 105 at a specific distance from one another. FIG. 7 depicts the PAS clamp 100 being used within an environment 700. FIG. 8 depicts a zoomed in view of the PAS clamp 100 of FIG. 7 having a vice as the attachment device 103. FIG. 9 illustrates how the PAS clamp 100, catheter/probe 706, and sheath 908 of a patient might be combined to create a system that allows better control of the catheter/probe 706 by a medical professional. FIG. 10 illustrates a method 800 that may be carried out by a user 705 using the PAS clamp 100. It is understood that the various method steps associated with the methods of the present disclosure may be carried out as operations by the user 705 depicted in FIG. 10.

As illustrated in FIGS. 1-9, the PAS clamp 100 generally comprises an attachment device 103, a bottom piece 105, bottom pad 106, top piece 110, top pad 111, hinge 115, and locking element 120. Other preferred embodiments of a PAS clamp 100 may include a plurality of bottom pieces 105, bottom pads 106, top pieces 110, top pads 111, hinges 115, and/or locking elements 120. In one preferred embodiment, as illustrated in FIG. 1, the PAS clamp 100 comprises a single bottom piece 105, bottom pad 106, top piece 110, bottom pad 106, hinge 115, and locking element 120. A catheter/probe 706 may be placed between the bottom pad 106 and top pad 111 of the PAS clamp 100. When said PAS clamp 100 is in the closed position 405, a friction force 300 created by the top pad 111 and bottom pad 106 is applied to the catheter/probe 706 in a way such that movement of the catheter/probe 706 is restricted in any direction. In some preferred embodiments, the locking element 120 may have several levels at which it can be locked to provide an adjustable amount of compression that allows it to accommodate various sizes of catheters as well as quick adjustment of tension during a procedure. In other preferred embodiments, the materials of the top pad 111 and/or bottom pad 106 may differ in compressibility or be textured in a way such that resistance in substantially one direction about a plane is created. Therefore, in some embodiments of a PAS clamp, if friction is produced by at least one of the top pad 111 or bottom pad 106 in substantially only the X-direction 300A, as illustrated in FIG. 3, the friction force 300 will mostly restrict radial movement of the catheter/probe 706, whereas if friction is produced by at least one of the top pad 111 or bottom pad 106 in substantially only the Z-direction 300B, the friction force 300 will mostly restrict linear movement of the catheter/probe 706.

The attachment device 103 is configured to secure the bottom piece 105 to an object that is at least semi-stationary. As such, the bottom piece 105 is preferably coupled to the attachment device 103. In a preferred embodiment, as illustrated in FIG. 1, the bottom piece 105 is coupled to the attachment device 103 in a way such that the attachment device 103 and bottom piece 105 form a single component. In a preferred embodiment, the attachment device 103 is a soft, flexible strip having an adhesive material thereon, wherein said adhesive material secures said strip and said bottom piece 105 to object chosen by a medical professional, such as the sterile patient drape. Alternatively, as illustrated in FIG. 8, the attachment device 103 may be a clamp that a medical professional may use to clinch to a stationary object to prevent the bottom piece 105 from moving. However, one with skill in the art may recognize that other attachment devices 103 configured to secure the bottom piece 105 to an object may be used without departing form the inventive subject matter described herein.

The top piece 110 and bottom piece 105 preferably comprise a rigid material suitable for applying force to a catheter/probe 706 situated therebetween, wherein said material maintains the shape of said top piece 110 and said bottom piece 105 while applying said force to said catheter/probe 706. As illustrated in FIG. 1, the top piece 110 and bottom piece 105 preferably comprise a generally rectangular shape; however, the top piece 110 and bottom piece 105 may comprise any shape suitable for maintaining tension on a catheter/probe 706 situated therebetween without departing from the inventive subject matter described herein. A hinge 115 is preferably located on one end of the top piece 110 and the bottom piece 105 with a locking element 120 situated on the opposite end of said top piece 110 and said bottom piece 105. The surface of the bottom piece 105 and the top piece 110 is configured to accept an adhesive that may be used to attach a bottom pad 106 and top pad 111 thereto, respectively. The material used to make the top piece, bottom piece, locking element, and hinge is preferably polymer, but one having skill in the art will recognize that any rigid material suitable for a clamp used in a healthcare environment can be used without departing form the inventive subject matter described herein.

The top pad 111 and bottom pad 106 are configured to apply friction to the catheter/probe 706 when said catheter/probe 706 is contained within the PAS clamp 100. The friction applied to the catheter/probe 706 is largely dependent on the attributes of the top pad 111 and bottom pad 106. For instance, if the top pad 111 and/or bottom pad 106 are less pliable, more force will be applied to the catheter/probe 706 by the top piece 110 and bottom piece 105 via the top pad 111 and bottom pad 106. For instance, a material having a higher friction coefficient will create more friction force 300 with less force applied by the top piece 110 and bottom piece 105 than a material having a lower friction coefficient. The material used to construct the bottom pad 106 and top pad 111 is at least partially compressible. In another preferred embodiment, the material used to construct the bottom pad 106 and top pad 111 has a high friction coefficient. In a preferred embodiment, the pads are constructed of soft rubber, silicon, or plastic; however, any pliable material suitable for holding a catheter/probe 706 may be used. Therefore, embodiments of the device may comprise of a plurality of materials having differing pliability and friction coefficients without departing from the inventive subject matter described herein.

Additionally, some embodiments of the PAS clamp 100 may comprise a top pad 111 and a bottom pad 106 comprised of at least two materials to create a desired pliability and friction force 300 when a catheter/probe 706 is contained therein. For instance, a top pad 111 and bottom pad 106 may comprise an outer layer of rubber and an inner layer of a soft pliable material, wherein the outer layer of rubber is positioned to contact the catheter/probe 706 when placed between the top pad 111 and bottom pad 106. As mentioned previously, some embodiments of the PAS clamp 100 may comprise a material that produces friction in substantially one direction. Therefore, some embodiments of a PAS clamp may comprise a first pad and second pad comprising of an outer layer made of a material that produces friction in substantially one direction in order to create a PAS clamps 100 that applies a friction force 300 to a catheter/probe 706 in substantially one direction, as depicted in FIG. 3. Alternatively, some embodiments of a PAS clamp 100 may comprise a first pad having an outer layer made of a material that produces friction in a first direction and a second pad comprising a material that produces friction in a second direction that is substantially perpendicular to the first direction, allowing for the creation of PAS clamps 100 that allows medical professionals to adjust not only the amount of resistance being applied to a catheter/probe 706 but also the direction in which the catheter/probe 706 is allowed to move.

The top piece 110 and bottom piece 105 of the PAS clamp 100 are preferably rotatably attached to one another via a hinge 115. In a preferred embodiment, the hinge 115 comprises a male half 115B and a female half 115A, wherein the female half 115A has at least two female members having a space between for the male half 115B and wherein the male half 115B has two male members configured to fit within the female members. The male members preferably snap into the female members in a way such that the male half 115B resists being removed from the female half 115A once snapped into place. This configuration allows a medical professional to combine the male half 115B and female half 115A to form the hinge 115, which allows the male half 115B and female half 115A to rotate about one another. This configuration may also allow the top half and bottom half of the PAS clamp 100 to be packed and shipped in separate sterile packages; though, other preferred embodiments of the PAS clamp 100 may come with the hinge 115 preassembled, allowing for the shipment of the PAS clamp in a single sterile package, wherein said PAS clamp 100 resides a sterile cavity of said sterile packaging until it is needed by a medical professional. In order to maintain the aseptic quality of the PAS clamp 100 up to the point of use, the sterile packaging is preferably sealed once the various pieces of the PAS clamp 100 are placed therein. For instance, a fully assembled PAS clamp 100 may be placed in a sterile packaging and subsequently sealed in a sterile manner to ensure the aseptic qualities of the PAS clamp 100 prior to use in a medical environment. For instance, a top piece 110, top pad 111, and male half 115B of a hinge 115 may be placed in a sterile packaging and subsequently sealed in a sterile manner to ensure the aseptic qualities of the that half of the PAS clamp 100 prior to use in a medical environment.

In yet another preferred embodiment, as illustrated in FIG. 5, the female members of the female half 115A may contain an opening on the lateral side that allows for insertion of the male members of the male half 115B. Insertion of the male members into the female members may create a quick release hinge, and the male members and female members are preferably shaped such that rotating the top piece 110 about the quick release hinge and engaging the locking element 120 prevents the male members and female members from decoupling. In another preferred embodiment of a quick release hinge, the shape of the male members and female members are such that decoupling is only possible when the device is in a specific position, wherein said specific position is preferably an open position 410. The quick release hinge may allow the top piece 110 to be quickly and easily removed by a medical professional during a procedure if necessary. Other attachments and hinge mechanisms known in the art may also be utilized to create the hinges 115 without departing from the inventive subject matter described herein.

In yet another preferred embodiment of a quick release hinge, as illustrated in FIG. 5 (bottom) a male half 115B may be configured to have an opening 115C that slots into a protrusion of a female half 115A (not shown) on one of the female members, wherein said aforementioned openings of the female members, as illustrated in FIG. 5 (top and bottom), allow for the male half 115B to be slid from one side of the female half 115A through the first female member to the other side of the female half 115A to the second female member, wherein the opening of the male half 115B may be slotted into the protrusion of said second female member. A user preferably may assemble and disassemble the quick connect hinge of this embodiment by placing the male half 115B and female half 115A in an open position before sliding said pieces together in the manner described. Once assembled, the top half is configured to This may allow for quick and secure assembly and disassembly of the PAS clamp in a sterile environment.

The locking element 120 is configured to hold the top piece 110 relative to the bottom piece 105 in a closed position 405, wherein the top pad 111 and the bottom pad 106 exert pressure on a catheter/probe 706 located therebetween when the PAS clamp 100 is in said closed position 405. The locking element 120 is also designed to allow quick adjustability of the amount of force applied to the catheter/probe with a single hand while the position of the catheter/probe is adjusted with the other hand. In ap referred embodiment, a healthcare professional may push down on the top piece 110 of the device with a thumb/finger until the locking element 120 engages. The healthcare professional may then laterally manipulate a top portion 120A of the locking element 120 with a thumb/finger to disengage the locking element 120 from the top piece 110 to allow for quick adjustment or release. Apparatuses that may act as the locking element 120 include, but are not limited to, push-pull pins, clamps, barbed fasteners, set knobs, snap locks, spring buttons, and clutch locks, or any combination thereof.

In some preferred embodiments, the locking element 120 may be configured in a way such that a user 705 may choose a distance from a plurality of distances that said top piece 110 and bottom piece 105 will be located from one another when in said closed position 405, depending on a setting of the locking element 120 chosen by the user 705. For instance, as illustrated in FIG. 6, a user 705 may choose the upper most locking step of a locking element 120 having a plurality of locking steps 120B configured to hold a top surface of said top piece 110 in order to secure the PAS clamp 100 in a closed position 405 with the least amount of force. By allowing a user 705 to choose the amount of force exerted on a catheter/probe 706 by choosing the locking step, and thus the amount of friction force 300 applied thereto, the user 705 may have even greater control of movement of the catheter/probe 706 when held by said PAS clamp 100. A locking element 120 comprising a plurality of locking steps 120B may also allow for one PAS clamp to accommodate multiple different sizes of catheters/probes 706. For instance, the same device could be used to hold a small electrophysiology catheter in a first position while simultaneously securing a large intracardiac echocardiography probe in a second position. This could again be controlled with a single hand so that the other hand may be free to adjust the position of the catheter/probe 706.

FIG. 10 provides a flow chart 1000 illustrating certain, preferred method steps that may be used to carry out the method of controlling a catheter/probe 706 using the PAS clamp 100. Step 1005 indicates the beginning of the method. During step 1010, the medical professional obtains a bottom piece 105 having an attachment device 103, a top piece 110, and a catheter/probe 706. The medical professional may then combine a female member of one of the top piece 110 and bottom piece 105 and the male member of one of the bottom piece 105 and top piece 110 to create the hinge 115 and form the PAS clamp 100 during step 1015. Though some preferred embodiments of the system 100 may come with the top piece 110 and bottom piece 105 preassembled, obviating the need to combine the top piece 110 and bottom piece 105 during step 1015. With the PAS clamp 100 in hand, the medical professional may attach the PAS clamp 100 to a stationary object using the attachment device 103 during step 1020. Once secured to the stationary object, the medical professional may place the catheter/probe 706 between the top pad 111 and bottom pad 106 of the PAS clamp 100 during step 1025 and subsequently engage the locking element 120 during step 1030.

During step 1035, the medical professional must perform a query to determine whether they would like to move the catheter/probe 706 that is located within the patient by way of a sheath 908, as illustrated in FIG. 9. Depending on the results of the query, the medical professional may perform an action during step 1040. If the medical professional determines that it is not necessary to move the catheter/probe 706, the medical professional may proceed to the terminate method step 1055. If the medical professional determines that it is necessary to move the catheter/probe 706, the medical professional may disengage the locking element 120 during step 1045. The medical professional may then move the catheter/probe 706 during step 1050 before once again placing the catheter/probe 706 between the top pad 111 and bottom pad 106 prior to reengaging the locking element 120 during steps 825 and 830, respectively. The medical professional may then repeat step 1035 until said medical professional determines there is no longer a need to move the catheter/probe 706 before proceeding to terminate method step 1055.

Some embodiments of a PAS clamp 100 may comprise more than one top piece 110 and/or bottom piece 105, as illustrated in FIGS. 7-9, wherein said more than one top piece 110 and/or bottom piece 105 are configured to control movement of a catheter/probe 706 in only a specific direction. In such embodiments, a user 705 may need to perform a query to determine which locking element 120 must be disengaged in order to move the catheter/probe 706 in the desired manner. For instance, a PAS clamp 100 having a first pad configured to restrict movement about an X-axis and a second pad configured to only restrict movement about a Y-axis may allow a medical professional to control insertion, removal, and radial movement of the catheter/probe 706. Therefore, a medical professional wanting to insert or remove the catheter/probe 706 may only need to disengage the locking element 120 of the first pad using this embodiment of a PAS clamp 100. And a medical professional wanting to move the catheter/probe 706 in a radial manner may only need to disengage the locking element 120 of the second pad using this embodiment of a PAS clamp 100. Therefore, some embodiments of a catheter/probe 706 may require a medical professional to perform multiple queries so that they may minutely control the manner said catheter/probe 706 moves throughout a patient's body.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, materials, and arrangements of the parts and method stages which have been described and illustrated in order to explain the nature of this inventive subject matter can be made without departing from the principles and scope of the inventive subject matter.

What is claimed is:

1) A system for a catheter/probe clamp comprising:
an attachment device having an adhesive on one side,
wherein said adhesive is designed to secure said attachment device to an object,
a bottom piece attached to said attachment device,
wherein a bottom pad that is at least semi-compressible is attached to a top surface of said bottom piece,
a top piece having a top pad,
wherein said top pad is at least semi-compressible and is attached to a bottom surface of said bottom piece,
a hinge having a male half and a female half,
wherein said female half is located on a hinge end of one of said bottom piece and said top piece,
wherein said male half is located on said hinge end of one of said bottom piece and said top piece that does not have said female half,
a locking element of said bottom piece and said top piece,
wherein said locking element holds said bottom piece and said top piece in a closed position,
a sterile packaging having a sterile cavity,
wherein said attachment device, bottom piece, bottom pad, top piece, top pad, hinge, and locking element are contained within said sterile cavity,
wherein said attachment device, bottom piece, bottom pad, top piece, top pad, hinge, and locking element are sterilized prior to being placed within said sterile cavity,
wherein an opening of said sterile packaging is sealed once said attachment device, bottom piece, bottom pad, top piece, top pad, hinge, and locking element are placed within said sterile cavity.

2) The system of claim 1, wherein said sterile packaging comprises a first sterile packaging and a second sterile packaging, wherein said bottom piece, bottom pad, and said attachment device are contained within said first sterile packaging, wherein said top piece and top pad are contained within said second sterile packaging.

3) The system of claim 1, wherein said top piece comprises a first top piece and a second top piece, wherein said first top piece comprises a first top pad, wherein said second top piece comprises a second top pad, wherein said first top piece rotates about said hinge independently of said second top piece.

4) The system of claim 3, wherein said locking element comprises a first locking element and a second locking element, wherein said first locking element is configured to hold said first top piece and said bottom piece in said closed position, wherein said second locking element is configured to hold said second top piece and said bottom piece in said closed position.

5) The system of claim 4, wherein said first top pad is configured to produce friction substantially in a first direction, wherein said second top pad is configured to produce said friction substantially in a second direction, wherein said first direction and said second direction are in plane, wherein said first direction and said second direction are perpendicular.

6) The system of claim 4, wherein said bottom pad comprises a first bottom pad and a second bottom pad, wherein said first bottom pad is in hinged communication with said first top pad, wherein said second bottom pad is in hinged communication with said second top pad.

7) The system of claim 1, wherein said female half and said male half are removably attached and rotatably attached.

8) The system of claim 7, wherein an opening of a first female member of said female half allows for insertion of a first male member of said male half, wherein said opening of a second female member of said female half allows for insertion of a second male member of said male half.

9) The system of claim 8, wherein an open position allows said female half and said male half to be decoupled, wherein a closed position does not allow for said female half and said male half to be decoupled.

10) The system of claim 1, wherein said locking element further comprises a plurality of locking steps, wherein each locking step of said plurality of locking steps holds said top piece and said bottom piece in said closed position at a specified distance, wherein each locking step of said plurality of locking steps is configured to engage a top surface of said top piece.

11) A system for a catheter/probe clamp comprising:
an attachment device,
a bottom piece attached to said attachment device,
wherein a bottom pad of said bottom piece is configured to apply a friction force to a catheter/probe placed thereon,
a first top piece having a first top pad,
wherein said first top pad is configured to apply said friction force to said catheter/probe placed thereon,
a second top piece having a second top pad,
wherein said second top pad is configured to apply said friction force to said catheter/probe placed thereon,
a hinge having a male member and a female member,
wherein said hinge rotatably attaches said first top piece and said second top piece to said bottom piece,
wherein said first top piece rotates about said hinge independently of said second top piece,
a first locking element of said bottom piece,
wherein said first locking element holds said bottom piece and said first top piece in a closed position,
a second locking element of said bottom piece,
wherein said second locking element holds said bottom piece and said second top piece in said closed position.

12) The system of claim 11, wherein said first top pad is configured to produce friction substantially in a first direction, wherein said second top pad is configured to produce said friction substantially in a second direction, wherein said first direction and said second direction are in plane, wherein said first direction and said second direction are perpendicular.

13) The system of claim 11, wherein said bottom pad comprises a first bottom pad and a second bottom pad, wherein said first bottom pad is in hinged communication with said first top pad, wherein said second bottom pad is in hinged communication with said second top pad.

14) The system of claim 13, wherein at least one of said first top pad and said first bottom pad is configured to produce friction substantially in a first direction, wherein at least one of said second top pad and said second bottom pad is configured to produce said friction substantially in a second direction, wherein said first direction and said second direction are in plane, wherein said first direction and said second direction are perpendicular.

15) The system of claim 11, wherein said female half and said male half are removably attached and rotatably attached.

16) The system of claim 15, wherein an opening of a first female member of said female half allows for insertion of a first male member of said male half, wherein said opening of a second female member of said female half allows for insertion of a second male member of said male half.

17) The system of claim 16, wherein an open position allows said female half and said male half to be decoupled, wherein a closed position does not allow for said female half and said male half to be decoupled.

18) The system of claim 11, wherein said locking element further comprises a plurality of locking steps, wherein each locking step of said plurality of locking steps holds said top piece and said bottom piece in said closed position at a specified distance, wherein each locking step of said plurality of locking steps is configured to engage a top surface of said top piece.

19) A system for a catheter/probe clamp comprising:
an attachment device,
a bottom piece attached to said attachment device,
wherein a bottom pad of said bottom piece is configured to apply a friction force to a catheter/probe placed thereon,
a top piece having a top pad,
wherein said top pad is configured to apply said friction force to said catheter/probe placed thereon,
a hinge having a male half and a female half,
wherein said female half is located on a hinge end of one of said bottom piece and said top piece,
wherein said male half is located on said hinge end of one of said bottom piece and said top piece that does not have said female half,
wherein said female half and said male half are removably attached and rotatably attached,
wherein an opening of a first female member of said female half allows for insertion of a first male member of said male half,
wherein said opening of a second female member of said female half allows for insertion of a second male member of said male half,
wherein an open position allows said female half and said male half to be decoupled, and
wherein a closed position does not allow for said female half and said male half to be decoupled.

20) The system of claim 19, wherein said locking element further comprises a plurality of locking steps, wherein each locking step of said plurality of locking steps holds said top piece and said bottom piece in said closed position at a specified distance, wherein each locking step of said plurality of locking steps is configured to engage a top surface of said top piece.
